(19) United States
(12) Patent Application Publication
(10) Pub. No.: US 2011/0034414 A1
(43) Pub. Date: Feb. 10, 2011

(54) FLUOROALKYLOXY ALKANES, PROCESS FOR PRODUCTION AND USES THEREOF

(75) Inventors: Lino Conte, Piove Di Sacco (IT); Mauro Beccaro, Cadoneghe (IT); Enrico Bettini, Fiesso D'Artico (IT); Paolo Signori, Verona (IT); Alessandro Zaggia, Padova (IT)

Correspondence Address: Pearne & Gordon LLP, 1801 East 9th Street, Suite 1200, Cleveland, OH, 44114-3108, US

(73) Assignee: AL.CHI.MI.A. S.R.L., 35020 PONTE SAN NICOLO' (PADOVA) (IT)

(21) Appl. No.: 12/937,915

(22) PCT Filed: Apr. 28, 2008
(86) PCT No.: PCT/IT08/00293

§ 371 Date: Oct. 19, 2010

(51) Int. Cl. A61K 31/695; C07C 43/12; A61K 47/24; A61P 27/02; A61P 41/00

(52) U.S. Cl. 514/63; 568/683; 514/772

(57) ABSTRACT

The present invention relates to fluoroalkyloxy alkanes having formula (I): R_{F}—R₁—O—R₂ wherein: R_{F} is a linear or branched perfluoroalkyl group, having from 1 to 12, preferably from 2 to 8, carbon atoms; R₁ is a linear or branched, non-fluorinated alkylene group, having from 1 to 6, preferably from 2 to 4, carbon atoms, —R² is a linear or branched, non-fluorinated alkyl group, having from 1 to 12, preferably from 2 to 8, carbon atoms, possibly containing at least one ether bond —O— along the chain. The present invention further relates to a process for preparation and use thereof, possibly in mixture with at least one silicone oil, as medicaments in ophthalmology, in particular as tamponade liquids in an operation for the treatment of retinal detachment.

The present invention relates to fluoroalkyloxy alkanes, to their synthesis process and to the use thereof for medical applications, in particular as liquids in ophthalmic treatments.

It is known about the use of liquid perfluorocarbons in ophthalmology, e.g. as tamponade liquids in operations for the treatment of retinal detachment or as vitreous body substitutes (see for instance patent application EP 0 089 232 A2).

Thanks to their high density (generally above 1.6 g/cm³) and to their extremely low surface tension (below 25 mN/m), liquid perfluorocarbons allow a complete unfolding and a correct re-positioning of the retina so as to favor the re-adhesion thereof. Perfluorocarbons are temporarily chemically inert and physiologically biocompatible products, if completely fluorinated. As a matter of fact, the presence, if any, of —CHF—CF₂— groups can give rise to dehydrofluorination with HF formation, resulting in the subsequent reduction of biocompatibility. Moreover, after their action as tamponade liquids during surgery is ended, perfluorocarbons cannot be kept in contact with the retina for long, since the high density can give rise, in time, to phenomena of thinning of the lower retina. Therefore, the use of perfluorocarbons is recommended only intra-surgically. If left in the eye also after surgery, these should anyhow be removed and replaced with a permanent tamponade liquid, such as a silicone oil.

It is known about the use of silicone oils (in particular polydimethylsiloxane) as tamponade liquids during ophthalmic surgery. These products, however, have some limitations related in particular to the fact that silicone oils have too low a density, usually of about 0.97 g/cm³ and thus lower than, water density, and cannot ensure a sufficient pressure in the retina, especially the lower retina, since they tend to come to the surface of the aqueous medium constituting the vitreous body.

Recently, other fluorinated compounds have been proposed as liquids that can be used in ophthalmic applications.

For instance, U.S. Pat. No. 6,262,126 B1 describes partially fluorinated alkanes with formula R_{F}R_{H} or R_{F}R_{H}R_{F}, wherein R_{F} is a linear or branched perfluoroalkyl group and R_{H} is a linear or branched, saturated alkyl group. The number of carbon atoms in the perfluorinated moiety can vary from 1 to 20, whereas for the alkyl moiety the number of carbon atoms is usually from 3 to 20. These products are obtained by reaction of perfluoroalkyl iodides with alkenes or α,ω-dienes, with HI elimination, and subsequent hydrogenation in the presence of a platinum catalyst or by reaction with tributyltin hydride. These products have a density generally of from 1.1 to 1.7 g/cm³, thus lower than the density of perfluoroalkanes. Since they have a certain solubility in non-fluorinated compounds, they can be used as tamponade liquids in mixture with silicone oils, with the goal of obtaining a density slightly higher than water.

International patent application WO 2005/117850 describes partially fluorinated ethers and the use thereof as tamponade agents in mixture with a liquid silicone or a liquid perfluorocarbon. These partially fluorinated ethers have a specific gravity of from 1.1 to 1.5 and the following formula:

Rf(CH₂)ₙO(CH₂)ₙRf

wherein Rf is a fluorinated, monovalent, saturated organic C1-C4 group and n is 3 or 4. Decafluoro-di-n-pentyl ether (DFPE) is particularly preferred. Preferably, the mixture comprises from 10 to 20% of DFPE and from 80 to 90% of silicone oil.

The Applicant has faced the problem of finding liquids with high biocompatibility, high purity degree and suitable density values so that they can be used as liquids in ophthalmic treatments, in particular as tamponade liquids during surgical operations on the retina, which are able to exert a sufficient pressure during said operation so as to enable a correct repositioning and a sufficient adhesion of the retina to the adjacent eye components, and which can be retained at the same time inside the eye structure for an undetermined lapse of time without damaging the retina itself.

The Applicant has found that this aim and others of the invention can be achieved by way of fluoroalkyloxy alkanes as defined hereinbelow, which both meet the above requirements and have a high solubility in unfluorinated liquids, in particular silicone oils, so as to enable the preparation of mixtures in a wide range of concentrations, which are stable at working temperatures, as better described in the following, without showing phase separation.

According to a first aspect, the present invention therefore relates to fluoroalkyloxy alkanes having formula (I):

R_{F}—R₁—O—R₂ (I)

wherein:
R_{F} is a linear or branched perfluoroalkyl group, having from 1 to 12, preferably from 2 to 8, carbon atoms;
R₁ is a linear or branched, non-fluorinated alkylene group, having from 1 to 6, preferably from 2 to 4, carbon atoms;
R₂ is a linear or branched, unfluorinated alkyl group, haying from 1 to 12, preferably from 2 to 8, carbon atoms, possibly containing at least one ether bond —O— along the chain.

Preferably, R_{F} is a linear perfluoroalkyl group having formula CF₃(CF₂)ₙ—, wherein n is an integer from 1 to 12, more preferably from 2 to 8.

Preferably, R₁ is a linear alkylene group having formula —(CH₂)ᵣ—, wherein r is an integer from 1 to 6, preferably from 2 to 4.

Preferably, R₂ is a linear, non-fluorinated alkyl group having formula CH₃(CH₂)ₘ—, wherein m is an integer from 1 to 12, more preferably from 2 to 8.

Optionally, R₂ may contain at least one ether bond along the chain; for instance, R₂ can have the following formula:

—(CH₂O)ₚ—(CH₂CH₂O)_{q}—R₃

wherein: R₃ is —CH₃ or —C₂H₅; p is zero or an integer from 1 to 4; q is an integer from 1 to 4; the —CH₂O— and —CH₂CH₂O— groups being statistically distributed along the chain.

Particularly preferred fluoroalkyloxy alkanes having formula (I) are: CF₃(CF₂)₅CH₂CH₂O(CH₂)₄CH₃, CF₃(CF₂)₅CH₂CH₂O(CH₂)₂CH₃, CF₃(CF₂)₃CH₂CH₂O(CH₂)₄CH₃, and CF₃(CF₂)₃CH₂CH₂O(CH₂)₂CH₃.

Preferably, fluoroalkyloxy alkanes having formula (I) have a density which may vary from 1.2 to 1.5 g/cm³, measured at 20° C.

According a second aspect, the present invention relates to a process for preparing a fluoroalkyloxy alkane having formula (I), which comprises reacting a fluorinated alcohol having formula (II):

R_{F}—R₁—OH (II)

wherein R_{F} and R₁ are as defined above, with an alkyl halide having formula (III):

R₂—X (III)

wherein R₂ is as defined above and X is a halogen selected from Br and I, preferably Br.

Preferably, the reaction is carried out at a basic pH, for instance by adding an aqueous solution of a strong base, such as NaOH or KOH, or a tertiary amine, e.g. triethylamine, in a solvent comprising water and at least one at least partially water-soluble organic solvent, which is able to solubilize at least partially the fluorinated alcohol having formula (II). Example of suitable organic solvents are: acetone, N,N-dimethyl formamide, N-methylpyrrolidone, tetrahydrofurane.

In order to promote the reaction between the fluorinated alcohol having formula (II), which is at least partially soluble in the aqueous medium, and the alkyl halide having formula (III), which is basically insoluble in the aqueous medium, the reaction mixture is added with at least one phase transfer catalyst, e.g. a quaternary ammonium salt, in particular a tetraalkyl ammonium salt or a benzyltrialkyl ammonium salt, e.g. benzyltriethyl ammonium chloride.

The reaction is preferably carried out at a temperature of from 25° C. to 100° C., preferably from 40° C. to 70° C., for a period of time generally of from 2 to 40 hours, preferably from 4 to 10 hours.

At the end of the reaction, after separating the aqueous phase, the organic phase can be purified, e.g. by distillation. In alternative or in addition to distillation, the end product can be purified by passage in a chromatographic column containing, for instance, silica or alumina.

According to a further aspect, the present invention relates to a mixture of at least one fluoroalkyloxy alkane having formula (I) and at least one silicone oil.

Preferably, the aforesaid mixture comprises from 10% to 95% by weight, more preferably from 20% to 80% by weight, of at least one fluoroalkyloxy alkane having formula (I), and from 5% to 90% by weight, more preferably from 20 to 80% by weight, of at least one silicone oil.

Preferably, said at least one silicone oil has a viscosity of from 100 to 100,000 cS (centistokes), measured at 20° C. Preferably, said silicone oil is a polydimethylsiloxane.

According to a further aspect, the present invention relates to a fluoroalkyloxy alkane having formula (I) for use as a medicament, in particular in ophthalmology.

According a further aspect, the present invention relates to a fluoroalkyloxy alkane having formula (I) for use as a tamponade liquid in an operation for the treatment of retinal detachment.

According a further aspect, the present invention relates to a fluoroalkyloxy alkane having formula (I) for use as a vitreous body substitute. This use can be either permanent or temporary, usually for at least 12 months.

According a further aspect, the present invention relates to a fluoroalkyloxy alkane having formula (I) for use as an agent for oxygenating biological tissues.

According to a further aspect, the present invention relates to a fluoroalkyloxy alkane having formula (I) as a drug carrier.

According to a further aspect, the present invention relates to a mixture of at least one fluoroalkyloxy alkane having formula (I) and at least one silicone oil for use as a medicament in ophthalmology.

According to a further aspect, the present invention relates to a mixture of at least one fluoroalkyloxy alkane having formula (I) and at least one silicone oil for use as a tamponade liquid in an operation for the treatment of retinal detachment.

According to a further aspect, the present invention relates to a mixture of at least one fluoroalkyloxy alkane having formula (I) and at least one silicone oil for use as a vitreous body substitute.

According to a further aspect, the present invention relates to the use of a fluoroalkyloxy alkane having formula (I) for producing a medicament for use in ophthalmology.

According to a further aspect, the present invention relates to the use of a fluoroalkyloxy alkane having formula (I) for producing a tamponade liquid for use in an operation for the treatment of retinal detachment.

According to a further aspect, the present invention relates to the use of a fluoroalkyloxy alkane having formula (I) for producing a vitreous body substitute.

According to a further aspect, the present invention relates to the use of a mixture of at least one fluoroalkyloxy alkane having formula (I) and at least one silicone oil for producing a medicament for use in ophthalmology.

According to a further aspect, the present invention relates to the use of a mixture of at least one fluoroalkyloxy alkane having formula (I) and at least one silicone oil for producing a tamponade liquid for use in an operation for the treatment of retinal detachment.

According to a further aspect, the present invention relates to the use of a mixture of at least one fluoroalkyloxy alkane having formula (I) and at least one silicone oil for producing a vitreous body substitute.

The present invention will now be disclosed in greater detail by way of some working examples, which are provided as mere non-limiting examples of the scope of the invention.

### EXAMPLE 1

To a mixture, kept under stirring, of C₆F₁₃CH₂CH₂OH (252 g, 0.692 moles), tetrahydrofurane (350 ml) and cyclohexane (300 ml) at 25° C., an aqueous solution of NaOH (400 ml, 50%) was added drop-wise. After stirring for 2 hours, benzyltriethyl ammonium chloride (25 g, 0.11 moles) as phase transfer catalyst, and then 1-bromopentane (209 g, 1.38 moles) were added. The reaction mixture was kept under stirring at 40° C. for 40 hours, then at 70° C. for 8 hours. The resulting mixture was poured into water and the organic phase thus obtained was washed two times with water. After removing the fraction having a low boiling point, the raw product was subjected to distillation at reduced pressure, thus obtaining 215 g of C₆F₁₃CH₂CH₂O(CH₂)₄CH₃ (yield 72%, boiling point 110° C./6 mbar, n_{D}²⁰ 1.3385). Spectrographic data confirm the obtained structure: GC/MS m/z at 435 (M+H)⁺, 377 (M−CH₂CH₂CH₂CH₃)⁺, 71 (—CH₂CH₂CH₂CH₂CH₃)⁺; for NMR data see Tables 1-2.

### EXAMPLE 2

To a mixture, kept under stirring, of C₆F₁₃CH₂CH₂OH (252 g, 0.687 moles), tetrahydrofurane (350 ml) and cyclohexane (300 ml) at 25° C., an aqueous solution of NaOH (400 ml, 50%) was added drop-wise. After stirring for 2 hours, benzyltriethyl ammonium chloride (25 g, 0.11 moles) as phase transfer catalyst, and then 1-bromopropane (170 g, 1.38 moles) were added. The reaction mixture was kept under stirring at 40° C. for 40 hours, then at 70° C. for 8 hours. The resulting mixture was poured into water and the organic phase thus obtained was washed two times with water. After removing the fraction having a low boiling point, the raw product was treated with toluene 2,4-diisocyanate, in order to remove unreacted fluorinated alcohol, and then subjected to distillation at reduced pressure, thus obtaining 200 g of C₆F₁₃CH₂CH₂—O—(CH₂)₂CH₃ (yield 71%, boiling point 174° C., n_{D}²⁰ 1.3245). Spectrographic data confirm the obtained structure: GC/MS m/z at 405 (M−H)⁺, 377 (M−CH₂CH₃)⁺, 73 (—CH₂OCH₂CH₂CH₃)⁺, 43 (—CH₂CH₂CH₃)⁺; for NMR data see Tables 1-2.

### EXAMPLE 3

To a mixture, kept under stirring, of C₄F₉CH₂CH₂OH (200 g, 0.757 moles), tetrahydrofurane (350 ml) and cyclohexane (300 ml) at 25° C., an aqueous solution of NaOH (400 ml, 50%) was added drop-wise. After stirring for 2 hours, benzyltriethyl ammonium chloride (25 g, 0.11 moles) as phase transfer catalyst, and then 1-bromopentane (200 g, 1.32 moles) were added. The reaction mixture was kept under stirring at 40° C. for 40 hours, then at 70° C. for 8 hours. The resulting mixture was poured into water and the organic phase thus obtained was washed two times with water. After removing the fraction having a low boiling point, the raw product was subjected to distillation at reduced pressure, thus obtaining 200 g of C₄F₉CH₂CH₂O(CH₂)₄CH₃ (yield 79%, boiling point 183° C., n_{D}²⁰ 1.3435). Spectrographic data confirm the obtained structure: GC/MS m/z at 335 (M+H)⁺, 277 (M−CH₂CH₂CH₂CH₃)⁺, 73 (—CH₂OCH₂CH₂CH₃)⁺, 43 (—CH₂CH₂CH₃)⁺; for NMR data see Tables 1-2.

### EXAMPLE 4

To a mixture, kept under stirring, of C₄F₉CH₂CH₂OH (200 g, 0.757 moles), tetrahydrofurane (350 ml) and cyclohexane (300 ml) at 25° C., an aqueous solution of NaOH (400 ml, 50%) was added drop-wise. After stirring for 2 hours, benzyltriethyl ammonium chloride (25 g, 0.11 moles) as phase transfer catalyst, and then 1-bromopropane (170 g, 1.38 moles) were added. The reaction mixture was kept under stirring at 40° C. for 40 hours, then at 70° C. for 8 hours. The resulting mixture was poured into water and the organic phase thus obtained was washed two times with water. After removing the fraction having a low boiling point, the raw product was subjected to distillation at reduced pressure, thus obtaining 200 g of C₄F₉CH₂CH₂O(CH₂)₂CH₃ (yield 86%, boiling temp. 147° C., n_{D}²⁰ 1.3255). Spectrographic data confirm the obtained structure: GC/MS m/z at 305 (M−H)⁺, 377 (M−CH₂CH₃)⁺, 73 (—CH₂OCH₂CH₂CH₃)⁺, 43 (—CH₂CH₂CH₃)⁺; for NMR data see Tables 1-2.

### EXAMPLE 5

The same fluoroalkyloxy alkanes as in Examples 1-4 were prepared following an alternative procedure. A mixture comprising 0.1 moles of fluorinated alcohol, 60 ml of N-methyl-2-pyrrolidone, 0.2 moles of 1-bromoalkane, and 60 ml of a 45% aqueous solution of KOH was heated under stirring for 5 hours at 50° C., then at 70° C. for 2 hours so as to complete the reaction. The raw reaction mixture was filtered so as to remove the formed KBr, and then diluted in 20 ml of water; the organic phase thus obtained comprised the desired fluoroalkyloxy alkane (yield: 80%).

As far as NMR data are concerned, these were obtained in acetone-d6. Indexes identifying the various carbon atoms are assigned as disclosed above for the product CF₃(CF₂)₃CH₂CH₂O(CH₂)₂CH₃:
CF₃(a)-CF₂(b)-CF₂(c)-CF₂(d)-CH₂(y)-CH₂(x)-O—CH₂(γ)-CH₂(β)-CH₃(α)

TABLE 1
(¹⁹F NMR DATA)

| Product | CF₃ (a) | CF₂ (b) | CF₂ (c) | CF₂ (d) | CF₂ (e) | CF₂ (f) |
|---|---|---|---|---|---|---|
| CF₃(CF₂)₃CH₂CH₂O(CH₂) | −84.2 t | −128.9 m | −127.3 m | −116.2 m | — | — |
| ₂CH₃ | (3F) | (2F) | (2F) | (2F) | | |
| CF₃(CF₂)₃CH₂CH₂O(CH₂) | −84.2 t | −128.9 m | −127.4 m | −116.3 m | — | — |
| ₄CH₃ | (3F) | (2F) | (2F) | (2F) | | |
| CF₃(CF₂)₅CH₂CH₂O(CH₂) | −83.9 t | −129.1 m | −126.3 m | −125.6 m | −124.6 m | −116.0 m |
| ₂CH₃ | (3F) | (2F) | (2F) | (2F) | (2F) | (2F) |
| CF₃(CF₂)₅CH₂CH₂O(CH₂) | −84.0 t | −129.1 m | −126.4 m | −125.6 m | −124.6 m | −116.0 m |
| ₄CH₃ | (3F) | (2F) | (2F) | (2F) | (2F) | (2F) |

TABLE 2
(¹H NMR DATA)

| Product | CH₃ (α) | CH₂ (β) | CH₂ (γ) | CH₂ (δ) | CH₂ (ε) | CH₂ (x) | CH₂ (y) |
|---|---|---|---|---|---|---|---|
| CF₃(CF₂)₃CH₂CH₂O(CH₂) | 0.90 | 1.56 | 3.41 | — | — | 3.73 | 2.49 |
| ₂CH₃ | t (3H) | tq (2H) | t (2H) | | | t (2H) | ttt (2H) |
| CF₃(CF₂)₃CH₂CH₂O(CH₂) | 0.89 | 1.32 | | 1.56 | 3.45 | 3.73 | 2.49 |
| ₄CH₃ | t (3H) | m (4H) | | m (2H) | t (2H) | t (2H) | ttt (2H) |
| CF₃(CF₂)₅CH₂CH₂O(CH₂) | 0.90 | 1.55 | 3.42 | — | — | 3.74 | 2.50 |
| ₂CH₃ | t (3H) | tq (2H) | t (2H) | | | t (2H) | ttt (2H) |
| CF₃(CF₂)₅CH₂CH₂O(CH₂) | 0.90 | 1.35 | 1.33 | 1.57 | 3.46 | 3.73 | 2.48 |
| ₄CH₃ | t (3H) | m (4H) | m (2H) | m (2H) | t (2H) | t (2H) | ttt (3H) |

### EXAMPLE 6

Various mixtures comprising a fluoroalkyloxy alkane obtained according to Examples 1, 3 and 4 and a silicone oil having a viscosity of 1,000 cS (at 20° C.) were prepared.

Table 3 contains the amounts of fluoroalkyloxy alkane in the various mixtures (expressed as weight % on the total weight of the mixture), and the result in terms of clarity of the solution at different temperatures.

TABLE 3

| Product | 20% | 33% | 43% | 50% | 60% | 90% |
|---|---|---|---|---|---|---|
| CF₃(CF₂)₅CH₂CH₂O | ++ | ++ | + | + | n.d. | n.d. |
| (CH₂)₄CH₃ | | | | | | |
| CF₃(CF₂)₃CH₂CH₂O | n.d. | ++ | ++ | ++ | ++ | ++ |
| (CH₂)₄CH₃ | | | | | | |
| CF₃(CF₂)₃CH₂CH₂O | n.d. | ++ | ++ | ++ | ++ | ++ |
| (CH₂)₂CH₃ | | | | | | |

++: the mixture is clear after stirring at room temperature
+: the mixture is opalescent after stirring at room temperature, it becomes clear after heating at 37° C.

### EXAMPLE 7

Various mixtures comprising a fluoroalkyloxy alkane obtained according to Examples 1, 3 and 4 and a silicone oil having a viscosity of 5,000 cS (at 20° C.) were prepared.

Table 4 contains the amounts of fluoroalkyloxy alkane in the various mixtures (expressed as weight % on the total weight of the mixture), and the result in terms of clarity of the solution at different temperatures.

TABLE 4

| Product | 20% | 33% | 43% | 50% | 60% | 90% |
|---|---|---|---|---|---|---|
| CF₃(CF₂)₅CH₂CH₂O | ++ | ++ | + | + | n.d. | n.d. |
| (CH₂)₄CH₃ | | | | | | |
| CF₃(CF₂)₃CH₂CH₂O | n.d. | ++ | ++ | ++ | ++ | ++ |
| (CH₂)₄CH₃ | | | | | | |
| CF₃(CF₂)₃CH₂CH₂O | n.d. | ++ | ++ | ++ | ++ | ++ |
| (CH₂)₂CH₃ | | | | | | |

++: the mixture is clear after stirring at room temperature
+: the mixture is opalescent after stirring at room temperature, it becomes clear after heating at 37° C.

### EXAMPLE 8

The following mixtures (weight %) comprising the fluoroalkyloxy alkanes obtained according to Examples 3 and 4 with silicone oils having different viscosity values were prepared:

TABLE 5

| | Mixture | Mixture | Mixture | Mixture |
|---|---|---|---|---|
| | 1 | 2 | 3 | 4 |
| CF₃ (CF₂) ₃CH₂CH₂O (CH₂) ₄CH₃ | 90 | 90 | — | — |
| CF₃ (CF₂) ₃CH₂CH₂O (CH₂) ₂CH₃ | — | — | 90 | 90 |
| Silicone Oil 1,000 cS | 10 | — | 10 | — |
| (20° C.) | | | | |
| Silicone oil 5,000 cS | — | 10 | — | 10 |
| (20° C.) | | | | |

The aforesaid mixtures were cooled at 4° C. After cooling, Mixtures 1-3 remained perfectly clear, whereas Mixture 4 showed a phase separation; after stirring opalescence was observed; after being brought to room temperature, Mixture 4 became clear after stirring.

These tests show the high solubility of fluoroalkyloxy alkanes according to the present invention with silicone oils in a wide range of concentrations, temperature and viscosity of said oils.

1-22. (canceled)

23. Fluoroalkyloxy alkanes having formula (I):
R_{F}—R₁—O—R₂ (I)
wherein:
R_{F} is a linear or branched perfluoroalkyl group, having from 1 to 12, preferably from 2 to 8, carbon atoms;
R₁ is a linear or branched, non-fluorinated alkylene group, having from 1 to 6, preferably from 2 to 4, carbon atoms;
R₂ is a linear or branched, non-fluorinated alkyl group, having from 1 to 12, preferably from 2 to 8, carbon atoms, possibly containing at least one ether bond —O— along the chain.

24. The fluoroalkyloxy alkanes according to claim 23, wherein:
R_{F} is a linear or branched perfluoroalkyl group having formula CF₃(CF₂)ₙ—, wherein n is an integer from 1 to 12, more preferably from 2 to 8; and/or
R₁ is a linear alkylene group having formula —(CH₂)ᵣ—, wherein r is an integer from 1 to 6, preferably from 2 to 4; and/or
R₂ is selected from a linear, non-fluorinated alkyl group having formula CH₃(CH₂)ₘ—, wherein m is an integer from 1 to 12, more preferably from 2 to 8, and a group having formula:
—(CH₂O)ₚ—(CH₂CH₂O)_{q}—R₃
wherein: R₃ is —CH₃ or —C₂H₅; p is zero or an integer from 1 to 4; q is an integer from 1 to 4; the —CH₂O— and —CH₂CH₂O— groups being statistically distributed along the chain.

25. The fluoroalkyloxy alkanes according to claim 23, selected from:
CF₃(CF₂)₅CH₂CH₂O(CH₂)₄CH₃,
CF₃(CF₂)₅CH₂CH₂O(CH₂)₂CH₃,
CF₃(CF₂)₃CH₂CH₂O(CH₂)₄CH₃, and
CF₃(CF₂)₃CH₂CH₂O(CH₂)₂CH₃.

26. The fluoroalkyloxy alkanes according to claim 23, having a density of from 1.2 to 1.5 g/cm³, measured at 20° C.

27. A process for preparing a fluoroalkyloxy alkane having formula (I):
R_{F}—R₁—O—R₂ (I)
wherein:
R_{F} is a linear or branched perfluoroalkyl group, having from 1 to 12, preferably from 2 to 8, carbon atoms;
R₁ is a linear or branched, non-fluorinated alkylene group, having from 1 to 6, preferably from 2 to 4, carbon atoms;
R₂ is a linear or branched, non-fluorinated alkyl group, having from 1 to 12, preferably from 2 to 8, carbon atoms, possibly containing at least one ether bond —O— along the chain;
comprising reacting a fluorinated alcohol having formula (II):
R_{F}—R₁—OH (II)
wherein R_{F} and R₁ are as defined above, with an alkyl halide having formula (III):
R₂—X (III)
wherein R₂ is as defined above and X is a halogen selected from Br and I, preferably Br.

28. The process according to claim 27, wherein:
R_{F} is a linear or branched perfluoroalkyl group having formula CF₃(CF₂)ₙ—, wherein n is an integer from 1 to 12, more preferably from 2 to 8; and/or
R₁ is a linear alkylene group having formula —(CH₂)ᵣ—, wherein r is an integer from 1 to 6, preferably from 2 to 4; and/or
R₂ is selected from a linear, non-fluorinated alkyl group having formula CH₃(CH₂)ₘ—, wherein m is an integer from 1 to 12, more preferably from 2 to 8, and a group having formula:
—(CH₂O)ₚ—(CH₂CH₂O)_{q}—R₃
wherein: R3 is —CH₃ or —C₂H₅; p is zero or an integer from 1 to 4; q is an integer from 1 to 4; the —CH₂O— and —CH₂CH₂O— groups being statistically distributed along the chain.

29. The process according to claim 27, wherein the reaction is carried out at a basic pH in a solvent comprising water and at least one at least partially water-soluble organic solvent.

30. The process according to claim 28, wherein the reaction is carried out at a basic pH in a solvent comprising water and at least one at least partially water-soluble organic solvent.

31. The process according to claim 29, further comprising adding to the reaction mixture at least one phase transfer catalyst.

32. The process according to claim 30, further comprising adding to the reaction mixture at least one phase transfer catalyst.

33. The process according to claim 27, wherein the reaction is carried out at a temperature of 25° C. to 100° C., preferably of 40° C. to 70° C., for a period of time generally of from 2 to 40 hours, preferably from 4 to 10 hours.

34. A mixture of at least one fluoroalkyloxy alkane having formula (I) according to claim 23 and at least one silicone oil.

35. The mixture according to claim 34, comprising from 10% to 95% by weight, preferably from 20% to 80% by weight, of at least one fluoroalkyloxy alkane having formula (I), and from 5% to 90% by weight, preferably from 20% to 80% by weight, of at least one silicone oil.

36. The mixture according to claim 34, wherein said at least one silicone oil has a viscosity of from 100 to 100.000 cS (centistokes), measured at 20° C.

37. A mixture of at least one fluoroalkyloxy alkane having formula (I) according to claim 24 and at least one silicone oil.

38. A mixture of at least one fluoroalkyloxy alkane having formula (I) according to claim 25 and at least one silicone oil.

39. A medical method, in particular an opthalmological medical method, comprising the step of using an effective amount of at least a fluoroalkyloxy alkane or of a mixture of at least said fluoroalkyloxy alkane and at least one silicone oil, the fluoroalkyloxy alkane having formula (I):
R_{F}—R₁—O—R₂ (I)
wherein:
R_{F} is a linear or branched perfluoroalkyl group, having from 1 to 12, preferably from 2 to 8, carbon atoms;
R₁ is a linear or branched, non-fluorinated alkylene group, having from 1 to 6, preferably from 2 to 4, carbon atoms;
R₂ is a linear or branched, non-fluorinated alkyl group, having from 1 to 12, preferably from 2 to 8, carbon atoms, possibly containing at least one ether bond —O— along the chain.

40. The method according to claim 39, wherein:
R_{F} is a linear or branched perfluoroalkyl group having formula CF₃(CF₂)ₙ—, wherein n is an integer from 1 to 12, more preferably from 2 to 8; and/or
R₁ is a linear alkylene group having formula —(CH₂)ᵣ—, wherein r is an integer from 1 to 6, preferably from 2 to 4; and/or
R₂ is selected from a linear, non-fluorinated alkyl group having formula CH₃(CH₂)ₘ—, wherein m is an integer from 1 to 12, more preferably from 2 to 8, and a group having formula:
—(CH₂O)ₚ—(CH₂CH₂O)_{q}—R₃
wherein: R₃ is —CH₃ or —C₂H₅; p is zero or an integer from 1 to 4; q is an integer from 1 to 4; the —CH₂O— and —CH₂CH₂O— groups being statistically distributed along the chain.

41. The method according to claim 39, wherein the fluoroalkyloxy alkane is selected from:
CF₃(CF₂)₅CH₂CH₂—O—(CH₂)₄—CH₃,
CF₃(CF₂)₅CH₂CH₂—O—(CH₂)₂CH₃,
CF₃(CF₂)₃CH₂CH₂—O—(CH₂)₄—CH₃, and
CF₃(CF₂)₃CH₂CH₂—O—(CH₂)₂CH₃.

42. The method according to claim 39 wherein said effective amount is used as a tamponade liquid in an operation for the treatment of retinal detachment.

43. The method according to claim 40 wherein said effective amount is used as a tamponade liquid in an operation for the treatment of retinal detachment.

44. The method according to claim 41 wherein said effective amount is used as a tamponade liquid in an operation for the treatment of retinal detachment.

45. The method according to claim 39 wherein said effective amount is used as a vitreous body substitute.

46. The method according to claim 40 wherein said effective amount is used as a vitreous body substitute.

47. The method according to claim 41 wherein said effective amount is used as a vitreous body substitute.

48. The method according to claim 39 wherein said effective amount is used as an agent for oxygenating biological tissues.

49. The method according to claim 40 wherein said effective amount is used as an agent for oxygenating biological tissues.

50. The method according to claim 41 wherein said effective amount is used as an agent for oxygenating biological tissues.

51. The method according to claim 39 wherein said effective amount is used as a drug carrier.

52. The method according to claim 40 wherein said effective amount is used as a drug carrier.

53. The method according to claim 41 wherein said effective amount is used as a drug carrier.
